(19) United States
(12) Patent Application Publication
(10) Pub. No.: US 2004/0247528 A1
(43) Pub. Date: Dec. 9, 2004

(54) Therapeutic decorative object

(76) Inventor: Oriana Schoneberg, Orlando, FL (US)

Correspondence Address: Oriana Schoneberg, 4400 Raymar Drive, Orlando, FL, 32839, US

(21) Appl. No.: 10/501,000

(22) PCT Filed: Jan. 17, 2003
(86) PCT No.: PCT/US03/01535

Related U.S. Application Data
(60) Provisional application No. 60/349,732, filed on Jan. 22, 2002.

(51) Int. Cl. A61K 9/72

(52) U.S. Cl. 424/40

(57) ABSTRACT

A therapeutic apparatus (50, 62, 67, 70, 71, 75, 78, 79, 40, 44, 45, 46, 80, 90, 103, 104, 106, 115, 125, 132, 134, 140, 144, 151, 152, 162, 166, 175, 181, 191, 187) includes one or more therapeutic devices (59, 61, 55, 66, 69, 70, 74, FIGS. 14A-14G, 43, 85, 94, 98, 102, 108, 110, 118, 121, 122, 128, 145, 146, 149, 153, 161, 170, 167, 177, 183, 189, 192, 190), such as a vaporizer, a massage unit, heating or cooling units, which arc camouflaged in a decorative object or in a clothing item or in a utilitarian object, such as a piece of furniture, all of which are worn or located adjacent a person who can obtain the therapeutic benefits from the items. A decorative object (50, 67, 40, 80, 90) can be shaped to be worn by a person and have a therapeutic device camouflaged therein so that the therapeutic object acts on the individual while the decorative object is being worn. The same applies to clothing and appeal (44, 45, 46, 104, 106, 115, 132, 134, 144, 152, 151, 150, 140) including a ladies purse (115) which can have a plurality of therapeutic devices therein, such as an electric massager or vaporizer or a heating pack which can be held against a person without drawing attention to the person receiving the therapy. Therapeutic items can also be incorporated into furniture (71, 162, 163, 164, 166, 181, 187), such as tables or beds, to provide therapeutic action through a vaporizer or the like while a person is sitting adjacent the furniture.

### BACKGROUND OF THE INVENTION

The object has an exterior covering with a predetermined appearance, texture and feel that is psychologically attractive and camouflages its purposes. The object encloses multifunctional and multi-use therapeutic devices with interchangeable modules to provide drugless, naturopathic relief of tension, cramps, sleeplessness, musculoskeletal aches, stiffness, strains and sprains, minor burns, bumps and bruises, swelling, headaches, and the like by taking advantage of resources drawn from prior art therapeutic and naturopathic healing systems.

The present invention provides commercial articles to its users with therapeutic abilities to promote normal functioning of the body and mind, but does not display the utilitarian nature of the therapeutic devices which might cause embarrassment or be unappealing to a user. The invention provides a useful and pleasant article with multiple uses and functions for personalized use to provide relief for the individual's mind and body and to promote their well being and health.

The products include naturopathic toys, clothing, accessories, bedding, furniture, electronics and other decorative articles or combination thereof.

All embodiments have a decorative camouflaging cover configured to resemble a predetermined object. They may be shaped to conform to the proper alignment of the body to improve posture or to cover a predetermined area. The devices appear to the public as attractive and normal objects. Interchangeable modules allow therapeutic devices to be customized for an individual person and may include heating and/or cooling elements, an aromatic container, containers containing salts, seeds, herbs, grains, oils minerals etc., a body alignment element and/or magnets, bioelectromagnetic frequency elements (sound, light) ionizers, acupressure, massage, and other holistic elements.

The therapeutic elements can be interchangeable interior and exterior pockets or may be part of the object itself made out of appropriate therapeutic material. They may be used singularly or combined with one or more holders using a number of corresponding fastener systems or have it built into an object. In a ladies handbag, for instance, one side may have a removable and closed container filled with solid or liquid material, such as rice, flax seed, sand, healing stones, water magnets or bioelectromagnetic element or cold/hot gel packs, to provide heating or cooling. Dry rice, for instance, when heated, produces moist heat to relieve a number of aches and pains while radioactive volcanic sand can be applied to enhance circulation and relieve arthritic pains. Hot mud, called “fango”, such as used in spas, or liquids like mineral spring water combined with bioelectromagnetic energy and herbs using different heating or cooling methods can be applied as compresses or used for inhaling purposes. Moisture, aroma and radiation can be used in any combination to achieve the desired heated effect. The container may be heated or cooled externally to a desired temperature in a microwave or freezer or by chemical reaction or by electrically heating or the like. The invention may additionally utilize a thermostat with or without a dial and/or temperature gauge. The invention may also provide an aromatic essence holder with an insert located in the proximity of the heating element to provide combined effects of the heat with the essence. Containers in the decorative support may also contain magnets, massage, liquid soak naturopathic cloth, or acupressure elements. So, for example, one side of the ladies bag may be warmed or cooled as desired, the aroma holder at the top may be filled with the desired aroma essence, while the other side of the bag may have a container with magnets. The ladies bag can also be used with battery operated massage (vibrator) inserts or acupressure (plastic balls) inserted in a desired location, for example, against the lower back.

According to the invention there is provided a universal decorative object containing multi-use, multi-function and multi-purpose modules to enhance the quality of life for the user.

For children, therapeutic toys may be used to comfort, heal and to ensure good posture and health. They promote recovery after illnesses, accidents and traumas or even enhance learning abilities and good night sleep. The toys may be anatomically shaped in designs to improve posture combined with pleasing aromas to relieve cold and allergy symptoms and thermal applications to keep children warm while walking to school in winter containing naturopathic ingredients known to prevent and help the body naturally heal itself. Naturopathic toys are used to help balance the mind and systems of the body.

A toy may be any size, shape or color and made out of materials that can be heated, cooled or moisturized (directly or indirectly) and combined with natural elements like rice, sand, aromas, herbs, and magnets as already described.

A toy Koala bear, for instance, may be placed around the neck with its body anatomically shaped to cover the upper back, shoulders and neck area. It would be of non-flammable material filled with herbs, grains etc. and heated in a microwave for approximately 2 minutes, depending on the size and ingredient. An aromatic essence could also be added to it. Roman chamomile, for example, can be applied directly to the bear covering or placed in a small container with a filter insert.

For stomach discomforts or lower back pain, a flat large thermo-toy may be used. In addition, inserts containing magnets and/or battery operated vibrators could be inserted.

The present invention is also intended to cover objects used for natural alignment of the body to relax back muscles and relieve pressure points. It may be in a removable, washable, soft plush-like material. An elongated dolphin, for instance, made of anatomically designed or memory foam, would fit every contour of the body to support the head, neck, spine, knees, ankles and joints. It contains a removable filling made of sand, rice or a gel pack heated or cooled internally or externally with an aromatic insert to help promote sleep and relieve tension and stiffness. Pockets can be filled with magnets, herb saturated materials, or massage vibrators to help relieve complaints and to enhance healthy sleeping habits.

The present toys are generally filled with healing ingredients in separate inserts but can also be filled directly, depending on their use and application. The combination of different modules and the application of warming or cooling effects together with modules for smelling, touching, seeing, tasting or listening gives the present toy a special ability to comfort and help relieve aches and pain. It also promote healing, relaxation, and sleeping, while concealing the healing substances necessary to help its users. A therapeutic toy also gives a child something to hug, cuddle and squeeze with enjoyment.

Examples of therapeutic accessories include all kind of bags, pocketbooks, folders, briefcases, head coverings, hand and footwear (such as gloves, shoes, insoles), bands, belts and jewelry. Each resembles the actual intended use of a product with an additional benefit of some naturopathic abilities to comfort its users. All the embodiments comprise the same universal inserts and are adapted to its embodiment and the needs of the individual user. A ladies handbag is described as well as a head covering, insoles and shoes, bands and the like. Gloves filled with heated or cooled herbed essences and/or magnets and acupressure balls or bands may be applied to different parts of the body.

Another embodiment includes naturopathic bedding and applies the same principles. This includes naturopathic mattresses combining a pillow within the mattress body with heating and cooling elements. Integrating a warming blanket, and/or essence cartridge, and using orthopedic foam which may incorporate magnets and vibrators and/or herbal compresses at acupressure points may be attached with a screw-on/snap-on mechanism onto a blanket. Covers, chair inserts and anatomically shaped devices may be used as partial or whole body supports and provide needed relief for aches or tension after an injury.

Fashionable naturopathic clothing incorporating magnets, bioelectromagnetic elements (sound, light, ionizers) acupressure devices, herbal inserts, aroma, essence and hot/cold gel packs or tubes filled with special liquid that can be heated or cooled or the like also anticipated as being within the spirit and scope of the invention.

### SUMMARY OF THE INVENTION

A therapeutic apparatus includes one or more therapeutic devices, such as a vaporizer, a massage unit, heating or cooling packs, which are camouflaged in a decorative object or in a clothing item or in a utilitarian object, such as a piece of furniture, all of which are worn or located adjacent a person who can obtain the therapeutic benefits from the items. A decorative object can be shaped to be worn by a person and have a therapeutic device camouflaged therein so that the therapeutic object acts on the individual while the decorative object is being worn. The same applies to clothing including a ladies purse which can have a plurality of therapeutic devices therein, such as an electric massager or vaporizer or a heating elements which can be held against a person without drawing attention to the person receiving the therapy. Therapeutic items can also be incorporated into furniture, such as tables or beds, to provide therapeutic action through a vaporizer or the like while a person is sitting adjacent the furniture.

### BRIEF DESCRIPTION OF THE DRAWINGS

Other objects, features, and advantages of the present invention will be apparent from the written description and the drawings in which:

FIG. 1 is a sectional view of a portion of a decorative toy animal camouflaging a plurality of therapeutic devices;

FIG. 2 is a sectional view of the decorative plush toy of FIG. 1 mounted on an individual's back;

FIG. 3 is a perspective view of the decorative plush toy of FIGS. 1 and 2 on a person's back;

FIG. 4 is a perspective view of a therapeutic seating attachment for placing in a seat;

FIG. 5A is a perspective view of the therapeutic seat attachment of FIG. 4 being folded for portability;

FIG. 5B is a perspective view of the folded seat attachment of FIGS. 4 and 5A;

FIG. 6 is a perspective view of a person sitting in a plane seat with a therapeutic toy animal;

FIG. 7 is a perspective view of a decorative item incorporating a vaporizer, magnets, hot/cold elements and/or the like.

FIG. 8 is a perspective view of an individual having a therapeutic vest while sitting on the therapeutic seat attachment and having a therapeutic vest and neck wrap;

FIG. 9 is a perspective view of a therapeutic folded blanket;

FIG. 10 is a sectional view of a supporting therapeutic mattress with a plurality of therapeutic devices having an individual thereon;

FIG. 11 is a sectional view of another supporting therapeutic mattress having an individual thereon;

FIG. 12A is a perspective view of a therapeutic pillow for placing between the legs of the individual on the mattress of FIGS. 10 and 11;

FIG. 12B is a perspective view of a spine support;

FIG. 13A is a front perspective view of a plush toy animal having a zippered opening;

FIG. 13B is a side elevation of the plush toy of FIG. 13A;

FIGS. 14A-14F illustrate different therapeutic additions for the cavity of the plush animal of FIGS. 13A and 13B or any other therapeutic object;

FIG. 15 is a perspective view of a flexible polymer type tube;

FIG. 15B is a sectional view of the flexible polymer type tube of FIG. 15A filled with a gel;

FIG. 16A is a vest having the filled tubes of 15A and 15B incorporated therein;

FIG. 16B is a perspective view of a glove having the tubes of FIGS. 15A and 15B therein;

FIG. 16C is a perspective view of a scarf having the tubes of 15A and 15B incorporated therein;

FIG. 17 is an elevation of a baby on a blanket holding a therapeutic porpoise between it's legs and arms;

FIG. 18 is a sectional view of the porpoise of FIG. 17;

FIG. 19 is a sectional view taken through the closed container filled with liquid salt, gel or flax seed of FIG. 18;

FIG. 20 is an elevation of an individual on a mattress holding the therapeutic toy of FIGS. 17-19;

FIG. 21 is a front elevation of an individual holding the therapeutic toy of FIG. 17;

FIG. 22 is a diagrammatic view of a plurality of therapeutic devices camouflaged in a decorative wrap;

FIG. 23 is a sectional view of the therapeutic devices of 22;

FIG. 23A is a perspective view of the support for an essence for a vaporizer having a vaporizing liquid added thereto;

FIG. 24 is an elevation of a person's foot;

FIG. 25 is an elevation of the bottom of a person's foot;

FIG. 26 is a side elevation of a person's foot being positioned on an inner sole of a shoe having acupressure points thereon;

FIG. 27 is a top elevation of the inner sole with magnets and pressure nodules of FIG. 26;

FIG. 28 is an exploded perspective of the shoe with the inner sole and aromatic essence being added thereto;

FIG. 29 is an exploded perspective view of a sandal having pressure nodules positioned thereon;

FIG. 30 is an ankle band having magnets, pressure points, and massage apparatus and heat or cold added thereto;

FIG. 30A is a sectional view of the ankle band of FIG. 30;

FIG. 31 is a perspective view of a therapeutic hat;

FIG. 32 is a perspective view of the hat of FIG. 31 having a vaporizing liquid added thereto;

FIG. 33 is an exploded view of a hat of FIGS. 31 and 32;

FIG. 34 is a sectional view of different therapeutic layering for a band for use in the hat of FIGS. 31-33;

FIG. 35 is an exploded perspective view of a hat having a therapeutic band therein;

FIG. 36 is a side elevation of a person wearing a therapeutic hat of FIGS. 31-35;

FIG. 37 is a front elevation of a person sitting in a chair holding a therapeutic purse;

FIG. 38 is a sectional view of the therapeutic purse of FIG. 37;

FIG. 39 is a perspective view of the purse of FIGS. 37 and 38;

FIG. 40 is a perspective view of a therapeutic animal bed having an animal therein;

FIG. 41 is a perspective view of another animal bed having therapeutic devices therein;

FIG. 42 is a perspective view of a microwave having a microwavable dog vest being placed therein;

FIG. 43 is a perspective view of a dog having therapeutic clothing;

FIG. 44 is a perspective view of another dog having therapeutic clothing;

FIG. 45 is a side elevation of a horse having a therapeutic ankle wrap and cover;

FIG. 46 is a rear elevation of a person illustrating some of the acupressure points;

FIG. 47 is a front elevation of a person wearing athletic clothing having a plurality of therapeutic devices thereto;

FIGS. 47A and 47B illustrate the therapeutic clothing of FIG. 47;

FIG. 48 is a perspective view of a jacket having therapeutic devices therein and having an aromatic essence liquid added thereto;

FIG. 48A is a perspective view of a women's top having therapeutic devices therein;

FIG. 49 is a perspective view of a pair of pants having therapeutic devices therein;

FIG. 50 is a perspective view of a corner of a room having camouflaged therapeutic devices therein;

FIG. 51 is a perspective view of a wall unit having therapeutic devices formed therein;

FIG. 52 is a perspective view of a bolt of cloth having therapeutic devices formed therein;

FIG. 53 is a perspective view of a coffee table having therapeutic devices therein;

FIG. 54 is a perspective view of playing cards made out of therapeutic material having devices camouflaged therein; and

FIG. 54A is a perspective view of a chest board having chest pieces with therapeutic devices therein.

### DETAILED DESCRIPTION OF THE PREFERRED EMBODIMENTS

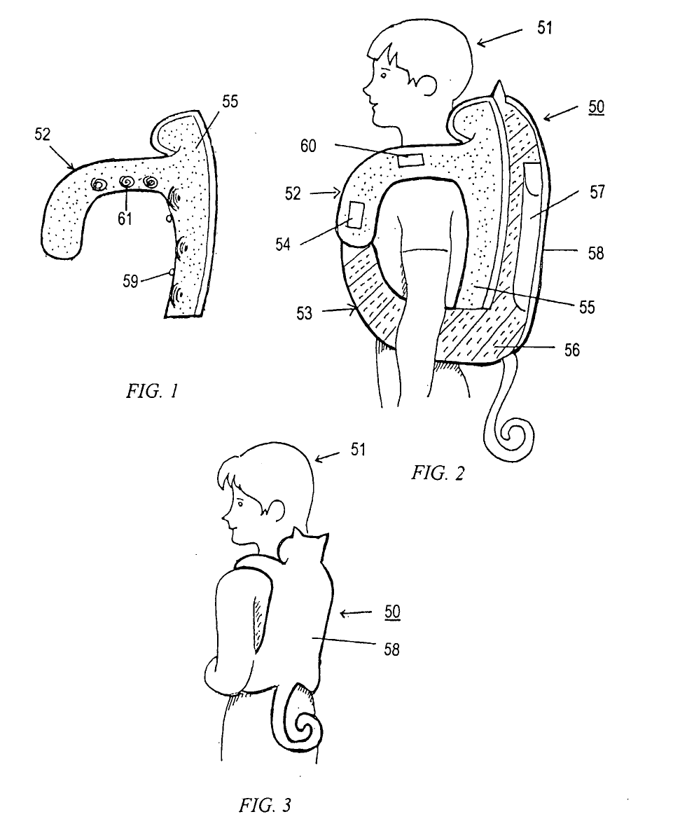

The invention is a decorative object (such as a toy, a game, picture, chair, a pullover, a hat, a wall, carpet, CD player, computer or music keyboard, with incorporated therapeutic devices for treating individuals without it appearing to others that they are obtaining treatment. As seen in FIGS. 1-54 and especially in FIGS. 1-3, a decorative toy animal 50 is shown on the back of a person 51 and is shown in the nature of a plush toy cat having upper arms 52 and lower arms 53 which allow the plush toy's therapeutic device 50 to fit onto a person's back similar to a backpack with the arms 52 and 53 wrapping around the shoulder and arms of the individual 51. The therapeutic toy 50 has fasteners 54 for holding the toy on the person's 51 back and includes an inner module 55 which can be a loose granular material or other material which will form fit to the individual's back and which, advantageously, allows heating or cooling of the material for radiating heat for a period after the material has been heated. Granular solids can include grains, beans, seeds, grasses, barks, or ordinary rice as well as sand, stone, salts and minerals and can also include water or oil mixes or gels for retaining the heat or the lack of heat for treating the individual. A foamed polymer material 56 can be form fitted around the module 55 and may be a foam having a memory to align with the body as well as to form an insulation layer over the module 55. The therapeutic toy 50 may have a storage pocket 57 as well as a reflective material cover 58 and might also include a holder 60 in which an aromatic essence and/or ions, sound etc. can be released into the air adjacent to the person's face. Additional treatment can be obtained with a plurality of magnets 61, as shown in FIG. 1, the area can also contain massage vibrators or ball-shaped devices or nodules 59 for applying pressure to the person's body while the person is walking, sitting and/or leaning against it. Advantageously, the therapeutic decorative object 50 can appear to be a toy while camouflaging its therapeutic values and a back pack for adults or vest for pets.

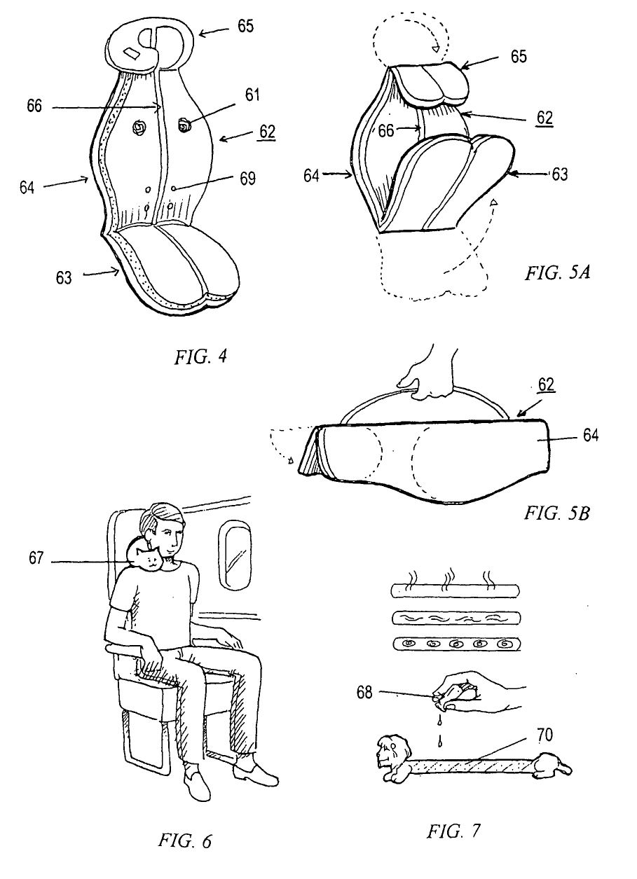

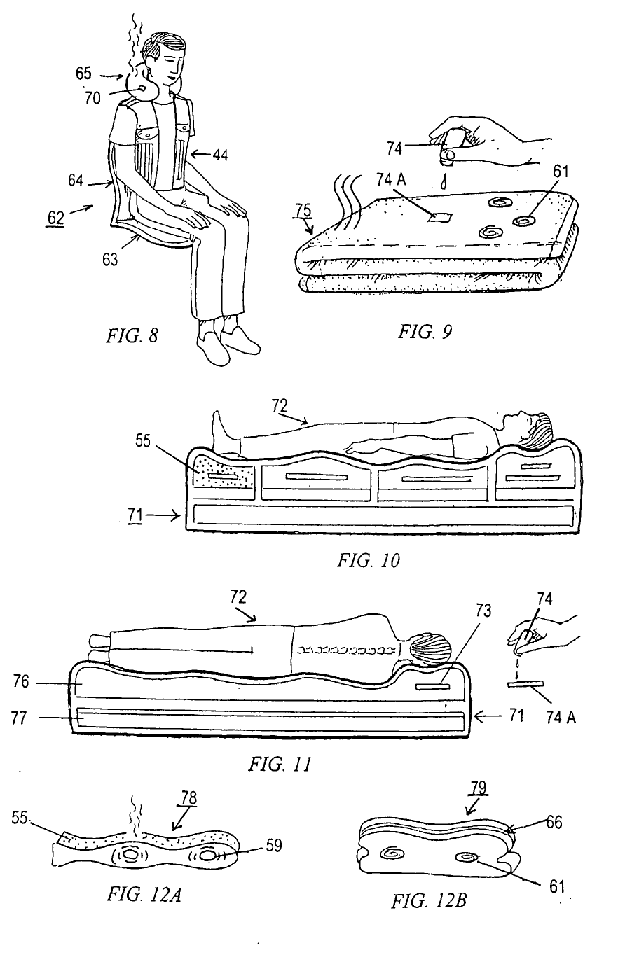

Turning to FIGS. 4-8, another embodiment of the therapeutic attachment is illustrated in the form of a seating attachment 62 which has a seat portion 63 and a back portion 64 as well as a neck wrap portion 65. The seating portion 63 and back portion 64 are spaced with a large groove 66. This allows a person sitting on the seat portion and leaning against the back portion, as seen in FIG. 6, to have their spine fit into the groove 66 to relieve pressure on the spine. The neck portion 65 can then wrap around a person's neck, as seen in FIG. 6, and may be a decorative toy animal 67 or the like. Advantageously, the seat object 62 can be folded, as seen in FIGS. 5A and 5B, for portability. The seat portion 63, the back portion 64, and the neck portion 65 can be enhanced with other modules, such as shown in FIG. 7, where the neck portion has a material 70 specially designed for receiving an aromatic or other chemical 68 which is dispensed and provides an aromatic essence around the person's head. In addition, as seen in FIG. 8, heating or cooling materials can be placed in the seat 63 and the back 64 and the neck 65 or 67 and permanent magnets placed therein and positioned, as desired, and acupressure ball-shaped nodules 69 can be positioned for pressing against pressure points of the individual. Massage vibrators can also be incorporated into the back or seat portion that can be activated through the pressure of the body or a switch. An individual in FIG. 8 is wearing a therapeutic vest 44.

Turning to FIGS. 9-12, a therapeutic blanket 75 is illustrated and can be heated or cooled, as well as have magnets 78 and can have an essence added thereto in a supporting material or directly on the blanket. A mattress 71 has the shape of a person's body, as illustrated in FIGS. 10 and 11. The mattress may be of one continuous piece, as shown in FIG. 11, or in therapeutic modular portions, as shown in FIG. 10. Each piece may be filled with different therapeutic ingredients. In addition, a special essence holding slot 73 is incorporated for receiving the aromatic essence 74 for the person 72 to breathe while he is resting or sleeping. Also, an absorbent material 74A, can have the essence applied thereto, as seen in FIG. 11, which can then be inserted into a slot 73 just beneath the individual's 72 head. FIGS. 14A-G has different therapeutic materials that can be used in the mattress 71.

As seen in FIG. 11, the form fitting mattress allows the spine of the individual 72 to maintain a straight line. FIG. 12A is a therapeutic pillow 78 having heated inserts 55 with massage vibrators 59 for placing between the legs of a person resting on the mattress. As shown in FIG. 12B, the back pillow 79 incorporates therapeutic devices that support the spine when a person is laying on their side. Also as shown in FIG. 11, the heated material 76, such as flax seed, rice, or grains, can provide a heat therapy to the person 72 and be heated by an electric blanket 77 that can be incorporated into the mattress surface as desired.

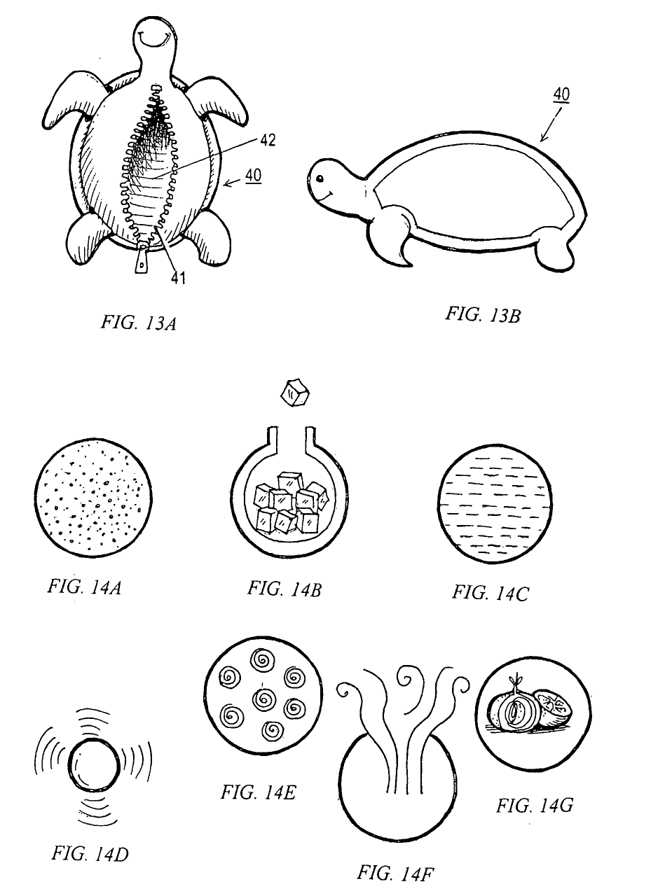

FIGS. 13A and 13B show a toy 40 shown as a turtle made of a plush, flexible material which is heat resistant. The toy 40 has a zipper 41 to allow entrance to the cavity 42. The cavity can have any of the inserts of FIGS. 14A-14G placed therein.

FIG. 14A shows loose granular material, such as grains, including flax seed and the like, which can be heated to retain heat in the toy of FIGS. 13A and 13B. FIG. 14B illustrate the use of ice in a waterproof container while FIG. 14C illustrates the use of liquid oils, gels and the like. FIG. 14D is a massage or vibrator while FIG. 14E shows a plurality of magnets. FIG. 14F is an ionizer, essence dispenser while FIG. 14G uses natural materials, such as onions or lemons. Any combination of FIGS. 14A-14G can be used in any one device.

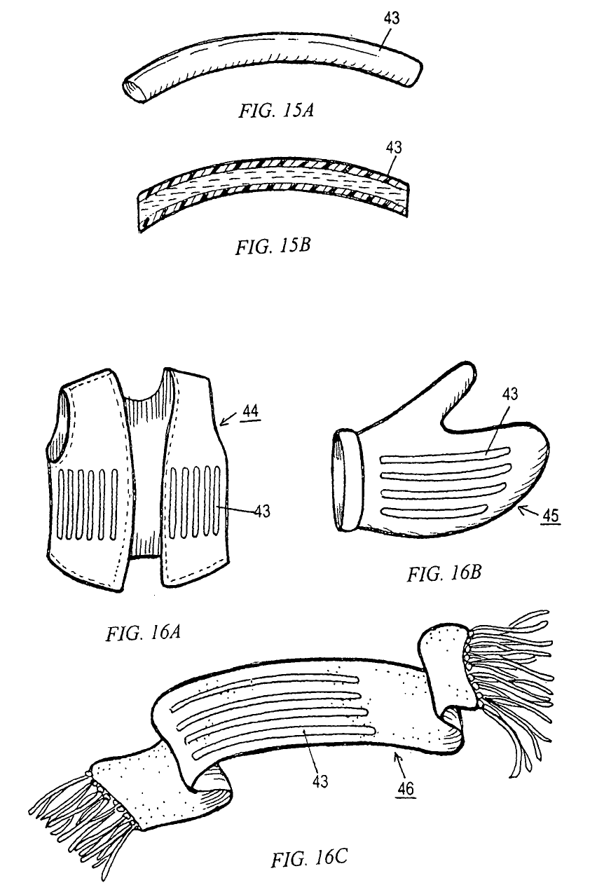

Turning to FIGS. 15A and 15B, a polymer type tube 43 is illustrated which will be used to hold the liquids, gels, grains and the like within the clothing of FIGS. 16A, 16B and 16C.

FIG. 16A shows a vest 44 having a plurality of the filled tubes 43 therein such that the whole vest can be heated in a microwave to heat or activate the ingredients in tubes 43 attached in the vest. Similarly, FIG. 16B is for a glove 45 having the filled tubes 43 mounted therein and FIG. 16C shows a scarf 46 having the flexible filled tube 43 therein. The fabric out of which the clothing is made can also have a loose granular heat holding material therein.

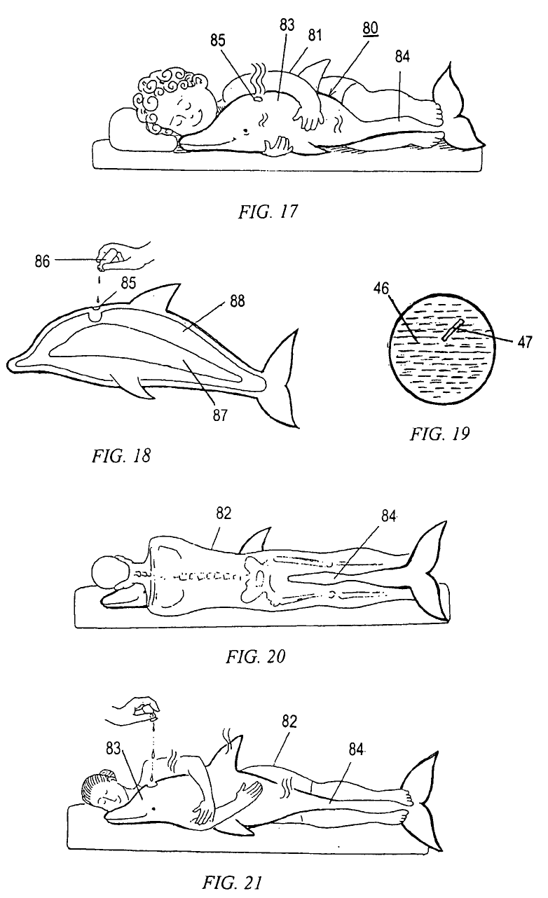

Turning to FIGS. 17, 18, 19, 20 and 21, another embodiment of a toy 80 is illustrated in the nature of a toy porpoise for a child 81 in FIG. 17 and supporting an adult 82 in FIG. 21. The porpoise is shaped with a head portion 83 for cuddling and support of the neck while a tail portion 84 is specifically shaped to provide support between the legs. In addition, an aromatic essence supporting material 85 is located in the toy's head for the individual 81 and 82 to breathe. A special heating material 87 can be placed within the middle of the therapeutic animal 80 and may be surrounded in a form fitting foam 88 or polyester fiber. FIG. 19 shows a heating liquid 46 in a sealed polymer material which can be activated by a trigger 47 placed therein. As seen in FIG. 20, the shape of the porpoise, when being held and supported, helps keep the spine aligned while providing the aromatic essence and heat to the individual 82 relieving tension, aches and e.g. cold symptoms.

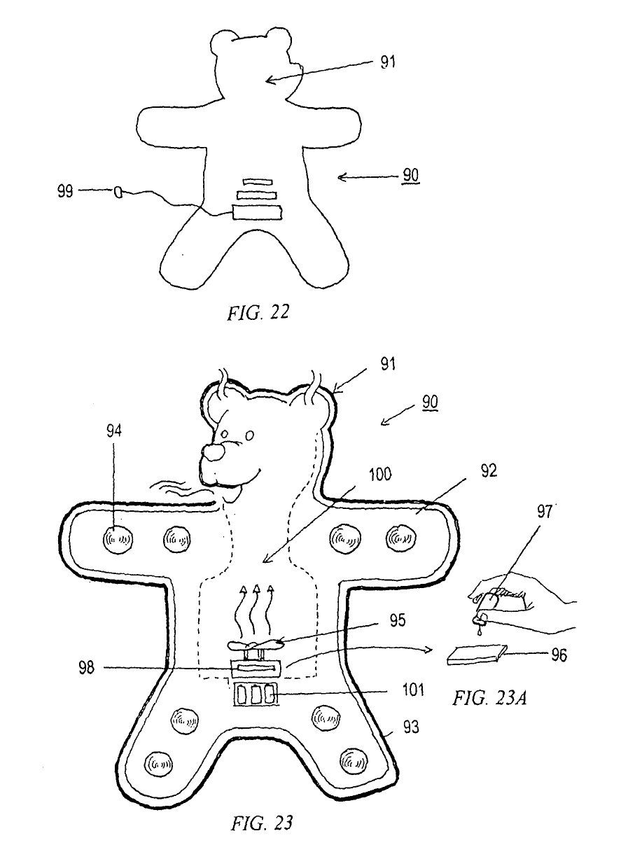

Turning now to FIGS. 22, 23 and 23A, a decorative teddy bear 90 of a flexible fabric body 93 has a decorative head 91 and arms 92, which can be wrapped around an individual or carried over their shoulder as desired. It may include massaging vibrators 94, as shown in FIG. 23, activated with a cord 99 and also may have an essence holder 96 for receiving an essence 97 for sliding into a slot 98 within the dispenser fan 95. Fan 95 may also be used for cooling. A hollowed out area 100 allows the essence to be distributed by the dispenser 95 which also has heating elements 101 for dispensing the essence and distributing cold air throughout the area of the therapeutic toy 90, such as a child's personal inhaler. It will, of course, be clear that magnets and other naturopathic elements at any layer within the decorative object body can also be distributed.

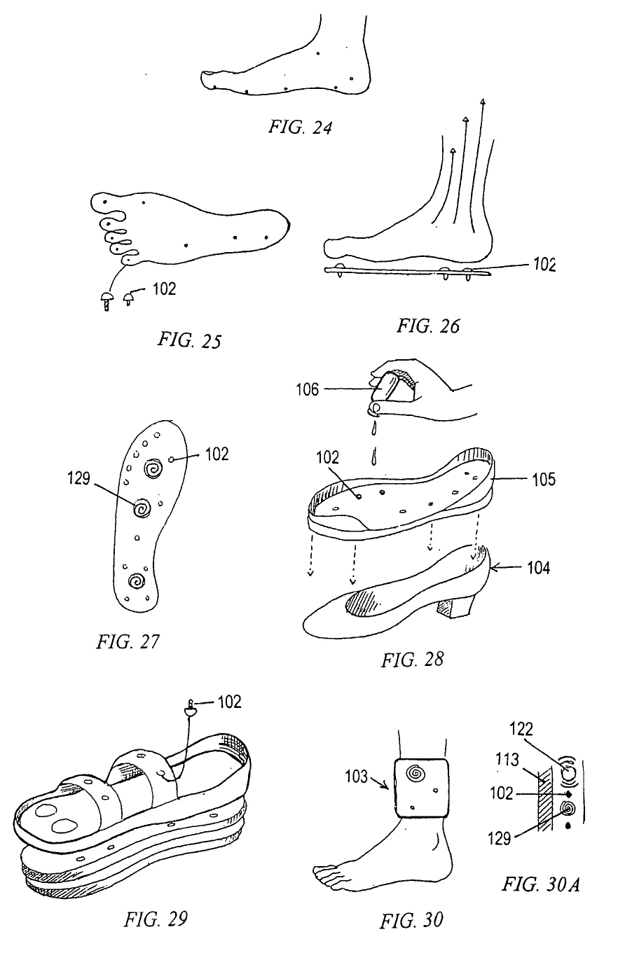

Turning to FIGS. 24-30, a therapeutic shoe and ankle band is illustrated in which acupressure points of a person's foot and ankle are shown, as illustrated in FIGS. 24 and 25, with the acupressure nodules 102 mounted to the base of a pad or shoe, as shown in FIGS. 26 and 27, apply pressure to the points, as illustrated in FIG. 24. The nodules 102 may be adjusted for height to adjust the pressure of the nodules against the skin surface. FIGS. 22, 30 and 30A also show magnets 129. In addition, the pressure points can be attached to an ankle band 103. The shoe 104 has an insert 105 in FIG. 28 which has both pressure points and an essence 106 holding surface on the pad 105 and it may also include acupressure elements, essence, magnets etc. as desired. The same is incorporated into a sandal as shown in FIG. 29. In FIGS. 30 and 30A, a band 103 has magnets 129 and pressure points 102 and a hot or cold element with essence supporting material 113 and massager 122.

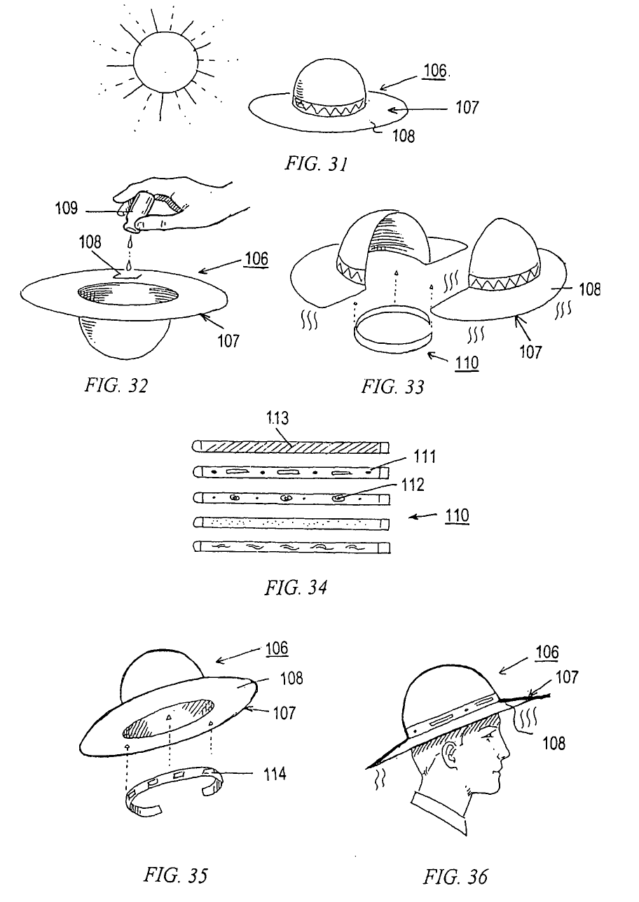

Turning to FIGS. 31-36, the therapeutic modules are incorporated into a hat 106 having a brim 107 with an absorbent essence holding surface 108. The essence 109 can be applied and positioned in front of a person's face for the evaporation and dispensing of the essence to a person directly over their nose, as seen in FIG. 36. Also available in the hat is a therapeutic band 110 which may have acupressure nodules 111 incorporated, along with magnets 112 and an essence supporting material 113, for holding the essence 109. The band can be filled with instant hot/cold packets or grains and herbs heated or cooled externally, or through the rays of the sun. The band can be attached, as shown in FIG. 36, with a VELCRO 114, as seen in FIG. 35.

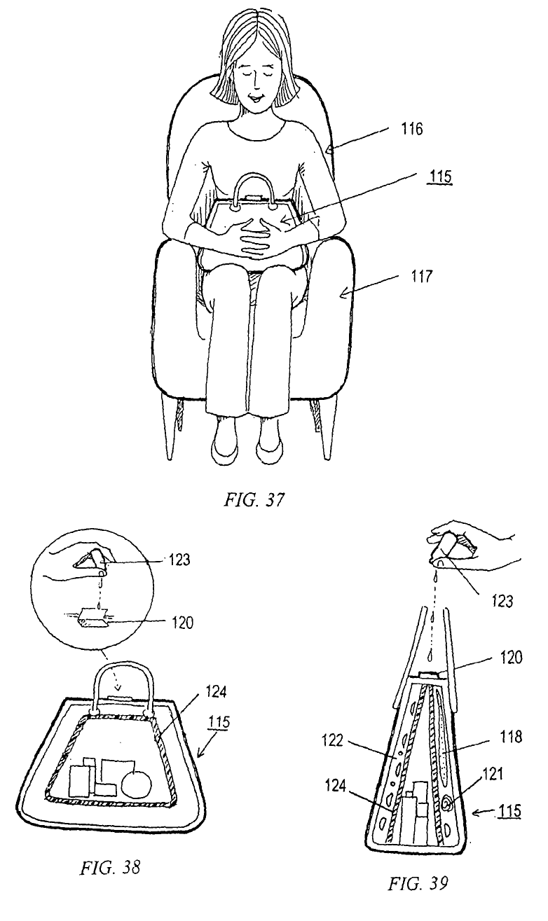

Turning to FIGS. 37-39, a handbag 115 is utilized for supporting the therapeutic modules and is held by a person 116 sitting in a chair 117, as seen in FIG. 37. The handbag can have heating and cooling material 118 on one side and may have an essence dispensing surface 120 on the top with an essence dispenser 123. The other side can support a massaging or vibrating surface 122 and can retain magnets 121, acupressure modules, or massage vibrators so the person holding the bag 115, as shown in FIG. 37, can hold the bag in either direction for obtaining a plurality of therapeutic treatments simultaneously even while traveling. The purse ingredients are contained in an insulated portion 124.

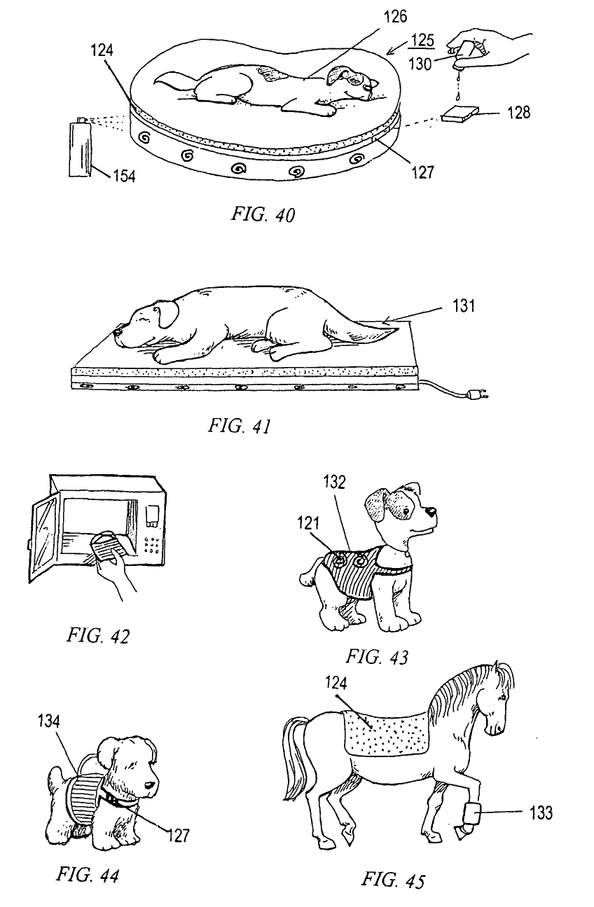

Turning to FIGS. 40-45, a therapeutic mattress 125 for an animal 126 is illustrated having a slot 127 for receiving an essence filter 128 for the essence 130. The mattress 125 for the animal 126 can also have a heating pad 131 and a layer of different granular materials 124, magnets and other therapeutic devices. FIG. 43 shows an individual dog wrapped with the supporting cover 132. An animal ankle wrap 133 is illustrated in FIG. 45 while a therapeutic wrap 134 is illustrated in FIG. 44. FIG. 42 shows the microwave heating of dog vest 132 or 134.

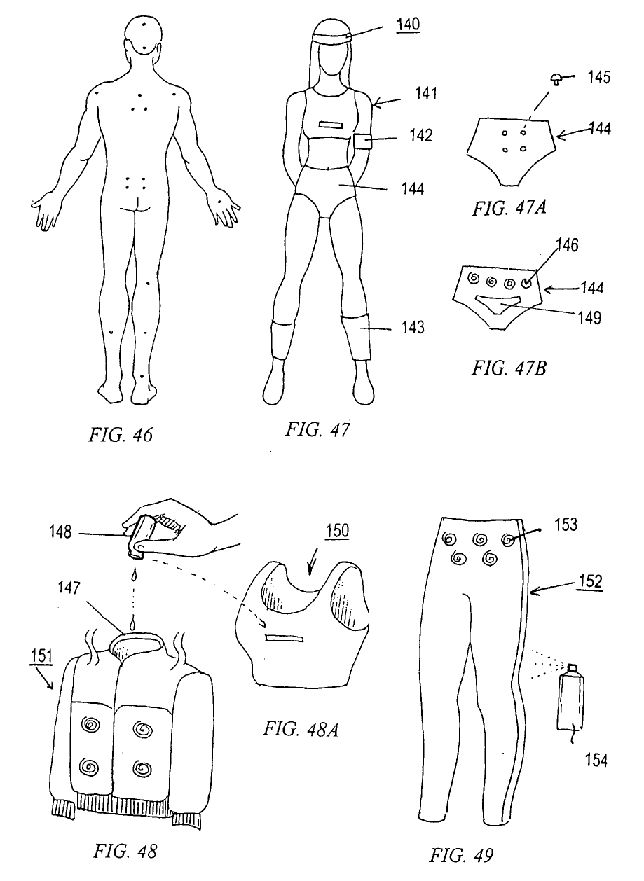

Referring to FIGS. 46-49, another embodiment is illustrated in which athletic decorative bands 140 can be on an athlete's 141 head while having a therapeutic module 142 wrapped around the person's arm and shin 143. Each of the wraps, as well as the athletic clothing 144, can have acupressure nodules 145 attached therein at specific points. They may also have magnets 146 positioned within the clothing and can also have a hot or cold pack 149 formed therein and a hot or cold pack therapeutic cloth may have a specially absorbent material 147 for receiving an essence 148 near the person's head in both a top 150 (FIG. 48A) and a jacket 151 (FIG. 48). Pants 152 may have pressure modules 153 as well as a sprayed on essence 154. The products include naturopathic jeans that help relive cellulite. Heated naturopathic gloves shawls and head covers. FIG. 46 illustrates some of the acupressure points in a person.

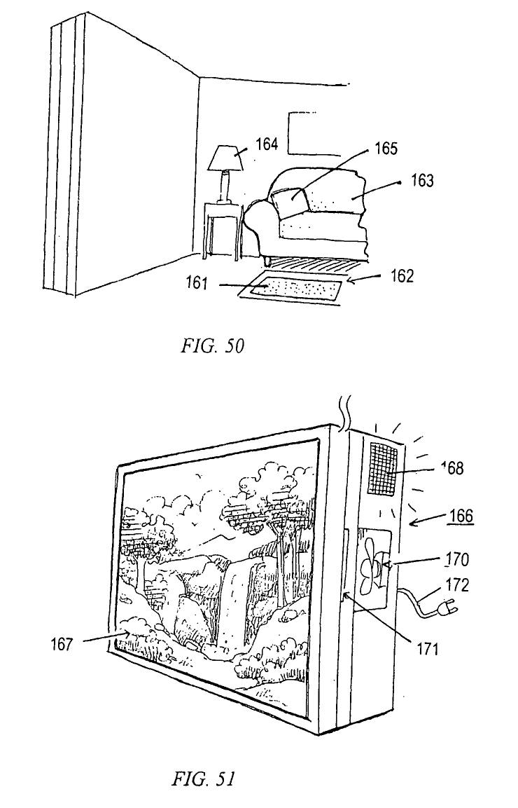

Referring to FIG. 50, a corner of a room 160 is illustrated having walls which may have incorporated therein therapeutic devices to give off radiation, such as light or sound radiations, magnetic fields and the like. Furniture in the room including the carpet 162 having a heating element 161 therein and a sofa 163 as well as the lamp 164, all of which can be provided with therapeutic covers which can be electrically heated and can contain or have added thereto naturopathic ingredients for dispensing heat with aromatic vapors and built in magnets. Pillow 165 may also contain moisture, magnets, electromagnetic fields and the like to provide a person with therapeutic aromas and naturopathic treatment while resting on the sofa. FIG. 51 illustrates a wall unit 166 which can be mounted to a wall or placed on a table and includes a relaxing scenic image 167 and a side mounted speaker 168 for delivering relaxing sound which can also be keyed to a changing image 167 or a light therapy. An air fan 170 can direct air through a filter 71 which may also have an aromatic essence placed therein or bioelectromagnetic fields for dispensing within the room. The therapeutic module 166 may have an electrical cord 172 for attaching to an electrical receptacle in a home.

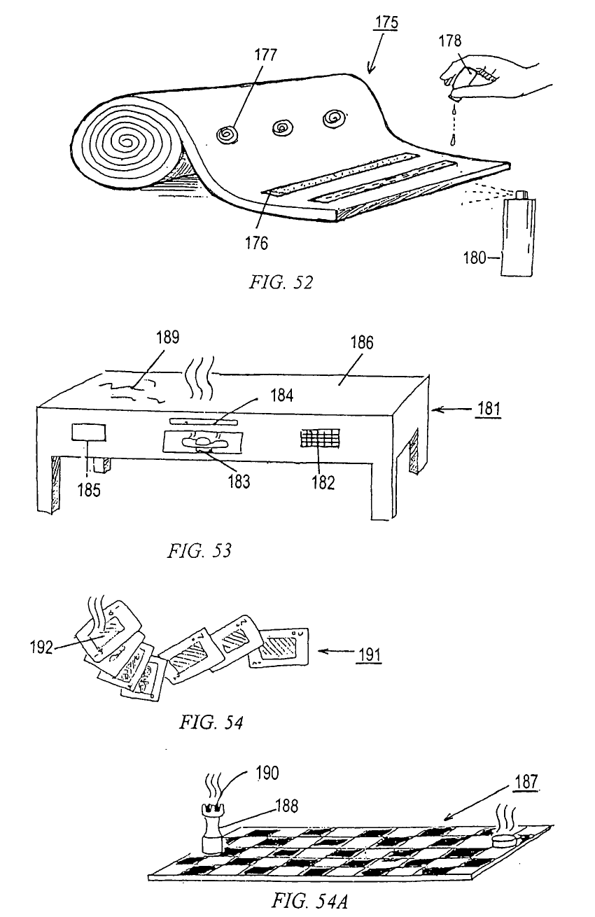

Referring to FIG. 52, a rolled-up pad of material 175 is illustrated having a plurality of tubes 176, such as the tubes 43 in FIGS. 15A and B and a plurality of magnets 177 or other inserts attached into the strips. The fabric can be made of a material for receiving therapeutic essence 178 thereinto for generating the inhalants or to relieve pain and can also be heated or cooled. The material 175 can be used to make clothes, furniture covers or as a mat to cover a surface but can also be cut and shaped and used to wrap toys, for instance, which can then be held by a person. The material can be sprayed with a spray 180 to provide a multi-therapeutic pad which can be taken with an individual while traveling.

FIG. 53 illustrates a table 181 having built-in therapeutic dispensing devices including a speaker 182 for dispensing pleasing therapeutic sounds or frequencies and an aromatic dispensing unit having a fan 183 directing air through a filter 184 having aromatic essences placed thereon for dispensing from openings in the table top or from a side air dispenser 185. The table top 186 can have an optical image 189 thereon which can be a continuously changing image or light designed for optical relaxation or be a source of heating or cooling.

FIG. 54A illustrates a chess board 187 having chess pieces 188 which have incorporated magnets heat and/or aromatic essence dispensers. Each chess piece 188 can have an essence supporting and dispensing material 190 with an aromatic essence material added thereto for dispensing while a person is playing chess. Similarly as seen in FIG. 54, a deck of playing cards 191 can be made out of non-flammable material and be warmed in a microwave prior to the game and have a dispensing surface 192 added thereto which can have an aromatic or other essence added thereto for dispensing into the air and body while a person is playing cards.

It should be clear at this time that a decorative object can be utilized as camouflage to cover a plurality of therapeutic treatment devices. The present invention is not to be limited to the forms shown but is to be considered illustrative rather than restrictive.

I claim:

1. A therapeutic apparatus (50,62,67,70, 71, 75,78,79,40,44,45,46,80,90,103,104,106,115,125,132, 134,140,144,151,152,162,166,175,181,191,187) comprising:
a therapeutic device (59,61,55,66,69,70,74, FIGS. 14A-14G,43,85,94,98,102,108,110,118,121,122, 128,145,146,149,153,161,167,170,177,183,189,192,190);
therapeutic device camouflage concealing said therapeutic device while said therapeutic device is located adjacent an individual receiving therapy from said therapeutic device whereby a person can receive therapy in the presence of other individuals without drawing attention to the therapeutic action taking place.

2. The therapeutic apparatus (50,62,67,70,71, 75,78,79,40,44,45,46,80,90,103,104,106,115,125,132, 134,140,144,151,152,162,166,175,181,191,187) of claim 1 including a plurality of therapeutic devices (59,61, 55,61,69,70,74, FIGS. 14A-14G,43,85,94,98,102,108, 110,118,121,122,128, 145,146,149,153,161,167,170,177, 183,189,192,190).

3. The therapeutic apparatus (50,62,67,70,71, 75,78,79,40,44,45,46,80,90,103,104,106,115,125,132, 134,140,144,151,152,162, 166,175,181, 191, 187) of claim 2 in which said plurality of therapeutic devices includes a vaporizer (70,74A,FIG. 14F,85,96, 108,120,128,147,161,183,188) for producing a therapeutic vapor adjacent a person.

4. The therapeutic apparatus (50,62,67,70,71, 75,78,79,40,44,45,46,80,90,103,104,106,115,125,132, 134,140,144,151,152,162,166,175,181,191,187) of claim 2 in which said plurality of therapeutic devices includes a massage unit (94,129,FIG. 14D,122) for producing a massaging vibration adjacent a person.

5. The therapeutic apparatus (50,62,67,70,71, 75,78,79,40,44,45,46,80,90,103,104,106,115,125,132, 134,140,144,151,152,162,166,175,181,191,187) of claim 2 in which said plurality of therapeutic devices includes heating means (FIGS. 14A and 14C, 43, FIG. 19,87, FIG. 42,131) including heat storing material (FIG. 14A).

6. The therapeutic apparatus (50,62,67,70,71, 75,78,79,40,44,45,46,80,90,103,104,106,115,125,132, 134,140,144,151,152,162,166,175,181,191,187) of claim 2 in which said plurality of therapeutic devices includes heating means of loose granular material which also forms a vaporizer when a vaporizing liquid is applied to the heated loose granular material.

7. The therapeutic apparatus (50,62,67,70,71, 75,78,79,40,44,45,46,80,90,103,104,106,115,125,132, 134,140,144,151,152,162,166,175,181,191,187) of claim 2 in which said camouflage is shaped like a soft sculpture animal figure (50,67,40,80,90) shaped to be attached to a person.

8. The therapeutic apparatus (50,62,67,70,71, 75,78,79,40,44,45,46,80,90,103,104,106,115,125,132, 134,140,144,151,152,162,166,175,181,191,187) of claim 1 in which said therapeutic device includes pressure points (69,59,102,145) placed camouflaged in clothing and positioned to apply pressure to predetermined points of a body.

9. The therapeutic apparatus (50,62,67,70,71, 75,78,79,40,44,45,46,80,90,103,104,106,115,125,132, 134,140,144,151,152,162,166,175,181,191,187) of claim 1 in which said therapeutic device includes means for generating a bioelectromagnetic field for applying to a person's body.

10. The therapeutic apparatus (50,62,67,70,71, 75,78,79,40,44,45,46,80,90,103,104,106,115,125,132, 134,140,144,151,152,162,166,175,181,191,187) of claim 1 in which said therapeutic device includes frequency generating means for generating a predetermined frequency adjacent the body.

11. A decorative therapeutic apparatus (50,67,40,80,90) comprising:
a decorative object shaped to be positioned adjacent to a person;
therapeutic means (59,61,55,66,69,70,74, FIGS. 14A-14G,43,85,94,98,102,108,110,118,121,122, 128,145,146,149,153,161,170,177,183,192,190) attached to said decorative object adjacent a person wearing said decorative object, said therapeutic means being camouflaged by said decorative object whereby camouflaged therapeutic means can provide a therapeutic action to a person wearing said decorative object.

12. The decorative therapeutic apparatus (50,67,40,80,90) of claim 11 in which said therapeutic means includes a plurality of therapeutic devices (59,61,55,61,69,70,74, FIGS. 14A-14G,43,85,94, 98,102,108,110,118,121,122,128,145,146,149,153, 161,170,177,183,192,190).

13. The decorative therapeutic apparatus (50,67,40,80,90) of claim 12 in which said therapeutic means includes a vaporizer (70,74,FIG. 14F,85,96) for producing a therapeutic vapor adjacent a person.

14. The decorative therapeutic apparatus (50,67,40,80,90) of claim 12 in which said therapeutic means includes a massage unit (94, FIG. 14D) for producing a vibration adjacent a person.

15. The decorative therapeutic apparatus (50,67,40,80,90) of claim 12 in which said therapeutic means includes heating means (FIGS. 14A and 14C,43,FIG. 19,87,FIG. 42,131) is loose granular material.

16. The decorative therapeutic apparatus (50,67,40,80,90) of claim 15 in which said loose granular material includes flax seeds.

17. The decorative therapeutic apparatus (50,67,40,80,90) of claim 12 in which said therapeutic means includes heating means of loose granular material which also form the vaporizer (FIG. 9) when a vaporizing liquid is applied to the heated loose granular material.

18. The decorative therapeutic apparatus (50,67,40,80,90) of claim 12 in which said therapeutic means includes a vaporizer having a heating element and a fan for blowing heated vaporizing liquid out of said decorative object.

19. The decorative therapeutic apparatus (50,67,40,80,90) of claim 12 in which said decorative object is shaped like a soft sculpture animal figure (50) shaped to be attached to a person.

20. The decorative therapeutic apparatus (50,67,40,80,90) of claim 12 in which said therapeutic means includes permanent magnets (61) mounted in said decorative object adjacent a person's body wearing said decorative object.

21. The decorative therapeutic apparatus (50,67,40,80,90) of claim 12 in which said therapeutic means includes pressure points (59) placed in said decorative object and positioned to apply pressure to predetermined points of a person's body.

22. A therapeutic clothing apparatus (44,45,46,104,106,115,132,134,144,152,151,150,140) comprising:
a clothing item (44,45,46,104,106,115,132, 134,144,152,151,150,140) for wearing by an individual;
a plurality of acupressure nodules, each located adjacent a selected known acupuncture point of an individual wearing said clothing item to apply pressure to a plurality of acupressure points of an individual wearing said clothing item.

23. The therapeutic clothing apparatus (44,45,46,104,106,115,132,134,144,152,151,150,140) of claim 22 including a plurality of magnets (61,49,146,121) located in said clothing item.

24. The therapeutic clothing apparatus (44,45,46,104,106,115,132,134,144,152,151,150,140) of claim 23 in which said clothing item is a shoe (104) having a plurality of acupressure nodules (102) positioned on the shoe insole.

25. A therapeutic clothing apparatus (106, 115,151) comprising:
a clothing item (44,45,46,104,106,115,132, 134,144,152,151,150,140) for wearing by an individual;
a camouflaged vaporizer attached to said clothing item positioned to direct a vapor adjacent an individual whereby an individual is exposed to a vapor while wearing said clothing item.

26. The therapeutic clothing apparatus (106,115,151) of claim 25 in which said clothing item is a hat having a brim having a passive vaporizer made of an vaporizing liquid support material formed in said brim to hold a vaporizing liquid for vaporizing adjacent a person's face and nose.

27. A therapeutic purse (115) comprising:
a ladies purse having a plurality of therapeutic devices (118,121,122) therein, said plurality of therapeutic devices including an electric massager (122) positioned for massaging an area of an individual's body when held thereagainst.

28. The therapeutic purse (115) in accordance with claim 27 in which said plurality of therapeutic devices includes a heating pad (118) positioned for heating an area of an individual's body when held thereagainst.

29. The therapeutic purse (115) in accordance with claim 28 in which said plurality of therapeutic devices includes a vaporizer (120) for vaporizing a liquid adjacent said purse when activated.

30. A therapeutic piece of furniture (71,162,163,164,166,181,187) comprising:
a piece of furniture (71,162,163,164,166, 181,187);
therapeutic means attached to said piece of furniture and being visually camouflaged from view whereby a therapeutic can provide therapeutic action on persons adjacent the furniture.

31. The therapeutic piece of furniture (71,162,163,164,166,181,187) in accordance with claim 30 in which said therapeutic means includes a plurality of therapeutic devices.

32. The therapeutic piece of furniture (71,162,163,164,166,181,187) in accordance with claim 31 in which said therapeutic means includes a vaporizer (183,190,170,161) for producing a therapeutic vapor adjacent said furniture.
